(19) United States
(12) Patent Application Publication
(10) Pub. No.: US 2024/0124458 A1
(43) Pub. Date: Apr. 18, 2024

(54) POLYMORPHS OF AVAPRITINIB AND METHODS FOR PREPARING THE POLYMORPHS

(71) Applicant: Macfarlan Smith Limted, Edinburgh (GB)

(72) Inventors: Thierry BONNAUD, Cambridge (GB); Zoe PRENTICE, Cambridge (GB)

(21) Appl. No.: 17/754,710

(22) PCT Filed: Oct. 23, 2020
(86) PCT No.: PCT/GB2020/052677

§ 371 Date: Apr. 8, 2022

(30) Foreign Application Priority Data
Oct. 24, 2020 (GB) 1915447.5

(51) Int. Cl. C07D 487/04; A61K 31/53

(52) U.S. Cl. CPC C07D 487/04; A61K 31/53; C07B 2200/13

(57) ABSTRACT

The present invention relates to crystalline and non-crystalline forms of avapritinib, to processes for their preparation, and to pharmaceutical compositions containing the crystalline or non-crystalline forms.

The present invention relates to crystalline and non-crystalline forms of avapritinib, to processes for their preparation, and to pharmaceutical compositions containing the crystalline or non-crystalline forms.

### BACKGROUND

Avapritinib has the IUPAC name of (1S)-1-(4-fluorophenyl)-1-[2-[4-[6-(1-methylpyrazol-4-yl)pyrrolo[2,1-f][1,2,4]triazin-4-yl]piperazin-1-yl]pyrimidin-5-yl]ethanamine and the chemical structure illustrated below:

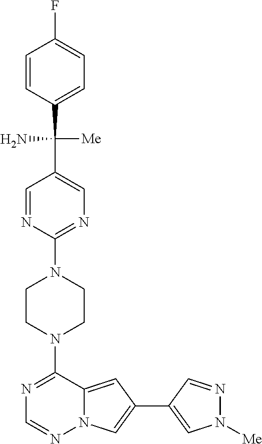 [SMILES: Cn1cc(-c2cc3c(N4CCN(c5ncc([C@@](C)(N)c6ccc(F)cc6)cn5)CC4)ncnn3c2)cn1]

In the EU, orphan designations have been granted for avapritinib for the treatment of gastrointestinal stromal tumours and for the treatment of mastocytosis.

WO2015/057873 (to Blueprint Medicines) relates to compounds and compositions useful for treating disorders related to the enzyme KIT and platelet-derived growth factor receptios (PDGFR). WO2015/057873 describes the synthesis of avapritinib.

Information about the solid-state properties of a drug substance is important. For example, different forms may have differing solubilities. Also, the handling and stability of a drug substance may depend on the solid form.

Polymorphism may be defined as the ability of a compound to crystallise in more than one distinct crystal species and different crystal arrangements of the same chemical composition are termed polymorphs. Polymorphs of the same compound arise due to differences in the internal arrangement of atoms and have different free energies and therefore different physical properties such as solubility, chemical stability, melting point, density, flow properties, hygroscopicity, bioavailability, and so forth. The compound avapritinib may exist in a number of polymorphic forms and many of these forms may be undesirable for producing pharmaceutically acceptable compositions. This may be for a variety of reasons including lack of stability, high hygroscopicity, low aqueous solubility and difficulty in handing.

### DEFINITIONS

The term “about” or “approximately” means an acceptable error for a particular value as determined by a person of ordinary skill in the art, which depends in part on how the value is measured or determined. In certain embodiments, the term “about” or “approximately” means within 1, 2, 3 or 4 standard deviations. In certain embodiments, the term “about” or “approximately” means within 30%, 25%, 20%, 15%, 10%, 9%, 8%, 7%, 6%, 5%, 4% , 3% , 2% , 1%, or 0.5% of a given value or range. In certain embodiments and with reference to X-ray powder diffraction two-theta peaks, the terms “about” or “approximately” means within ±0.2° 2θ.

The term “ambient temperature” means one or more room temperatures between about 15° C. to about 30° C., such as about 15° C. to about 25° C.

The term “amorphous” describes a solid which is not crystalline i.e. one that has no long-range order in its lattice (see, Oxford Dictionary of Chemistry, 6^{th} Edition, 2008).

The term “anti-solvent” refers to a first solvent which is added to a second solvent to reduce the solubility of a compound in that second solvent. The solubility may be reduced sufficiently such that precipitation of the compound from the first and second solvent combination occurs.

The term “consisting” is closed and excludes additional, unrecited elements or method steps in the claimed invention.

The term “consisting essentially of” is semi-closed and occupies a middle ground between “consisting” and “comprising”. “Consisting essentially of” does not exclude additional, unrecited elements or method steps which do not materially affect the essential characteristic(s) of the claimed invention.

The term “comprising” is inclusive or open-ended and does not exclude additional, unrecited elements or method steps in the claimed invention. The term is synonymous with “including but not limited to”. The term “comprising” encompasses three alternatives, namely (i) “comprising”, (ii) “consisting”, and (iii) “consisting essentially of”.

The term “crystalline” and related terms used herein, when used to describe a compound, substance, modification, material, component or product, unless otherwise specified, means that the compound, substance, modification, material, component or product is substantially crystalline as determined by X-ray diffraction. See, e.g., Remington: The Science and Practice of Pharmacy, 21st edition, Lippincott, Williams and Wilkins, Baltimore, Md. (2005); The United States Pharmacopeia, 23rd ed., 1843-1844 (1995).

The terms “polymorph,” “polymorphic form” or related term herein, refer to a crystal form of one or more molecules of avapritinib, or avapritinib molecular complex thereof that can exist in two or more forms, as a result different arrangements or conformations of the molecule(s) in the crystal lattice of the polymorph.

The term “pharmaceutical composition” is intended to encompass a pharmaceutically effective amount of avapritinib of the invention and a pharmaceutically acceptable excipient. As used herein, the term “pharmaceutical compositions” includes pharmaceutical compositions such as tablets, pills, powders, liquids, suspensions, emulsions, granules, capsules, suppositories, or injection preparations.

The term “excipient” refers to a pharmaceutically acceptable organic or inorganic carrier substance. Excipients may be natural or synthetic substances formulated alongside the active ingredient of a medication, included for the purpose of bulking-up formulations that contain potent active ingredients (thus often referred to as “bulking agents,” “fillers,” or “diluents”), or to confer a therapeutic enhancement on the active ingredient in the final dosage form, such as facilitating drug absorption or solubility. Excipients can also be useful in the manufacturing process, to aid in the handling of the active substance, such as by facilitating powder flowability or non-stick properties, in addition to aiding in vitro stability such as prevention of denaturation over the expected shelf life.

The term “patient” refers to an animal, preferably a patient, most preferably a human, who has been the object of treatment, observation or experiment. Preferably, the patient has experienced and/or exhibited at least one symptom of the disease or disorder to be treated and/or prevented. Further, a patient may not have exhibited any symptoms of the disorder, disease or condition to be treated and/prevented, but has been deemed by a physician, clinician or other medical professional to be at risk for developing said disorder, disease or condition.

The terms “treat,” “treating” and “treatment” refer to the eradication or amelioration of a disease or disorder, or of one or more symptoms associated with the disease or disorder. In certain embodiments, the terms refer to minimizing the spread or worsening of the disease or disorder resulting from the administration of one or more therapeutic agents to a patient with such a disease or disorder. In some embodiments, the terms refer to the administration of a molecular complex provided herein, with or without other additional active agents, after the onset of symptoms of a disease.

The term “overnight” refers to the period of time between the end of one working day to the subsequent working day in which a time frame of about 12 to about 18 hours has elapsed between the end of one procedural step and the instigation of the following step in a procedure.

### BRIEF DESCRIPTION OF THE FIGURES

Certain aspects of the embodiments described herein may be more clearly understood by reference to the drawings, which are intended to illustrate but not limit, the invention, and wherein:

FIG. 1 is a representative XRPD pattern of amorphous avapritinib.

FIG. 2 is a representative XRPD pattern of avapritinib anhydrate.

FIG. 3 is a representative TGA thermogram and a DSC thermogram of avapritinib anhydrate.

FIG. 4 is a representative GVS isotherm plot of avapritinib anhydrate. The solid black diamond symbol ( [symbol] ) represents the cycle 1 sorption isotherm plot. The solid grey symbol ( [symbol] ) represents the cycle 1 desorption isotherm plot. The solid grey triangle symbol ( [symbol] ) represents the cycle 2 sorption isotherm plot. The black cross symbol ( [symbol] ) represents the cycle 2 desorption isotherm plot. The grey star-like symbol ( [symbol] ) represents the cycle 3 sorption isotherm plot.

FIG. 5 is a representative XRPD pattern of avapritinib methanol solvate.

FIG. 6 is a representative TGA thermogram and a DSC thermogram of avapritinib methanol solvate.

FIG. 7 is a representative XRPD pattern of avapritinib hydrate.

FIG. 8 is a representative TGA thermogram and a DSC thermogram of avapritinib hydrate.

FIG. 9 is a representative GVS isotherm plot of avapritinib hydrate. The solid black diamond symbol ( [symbol] ) represents the cycle 1 sorption isotherm plot. The solid grey symbol ( [symbol] ) represents the cycle 1 desorption isotherm plot. The solid grey triangle symbol ( [symbol] ) represents the cycle 2 sorption isotherm plot. The black cross symbol ( [symbol] ) represents the cycle 2 desorption isotherm plot. The grey star-like symbol ( [symbol] ) represents the cycle 3 sorption isotherm plot.

### DESCRIPTION OF THE INVENTION

### Amorphous Avapritinib

It has been discovered that avapritinib can be prepared as an amorphous form. The avapritinib polymorph provided by the present invention is useful as an active ingredient in pharmaceutical formulations. In certain embodiments, the amorphous form is purifiable. In certain embodiments and depending on time, temperature and humidity, the amorphous form is stable. In certain embodiments, the amorphous form is easy to isolate and handle. In certain embodiments, the process for preparing the amorphous form is scalable. In certain embodiments, the amorphous form may exhibit a higher solubility as compared to a crystalline form.

The amorphous form described herein may be characterised using a number of methods known to the skilled person in the art, including X-ray powder diffraction (XRPD), differential scanning calorimetry (DSC), thermal gravimetric analysis (TGA), infrared spectroscopy, Raman spectroscopy, nuclear magnetic resonance (NMR) spectroscopy (including solution and solid-state NMR). The chemical purity may be determined by standard analytical methods, such as thin layer chromatography (TLC), gas chromatography, high performance liquid chromatography (HPLC), and mass spectrometry (MS).

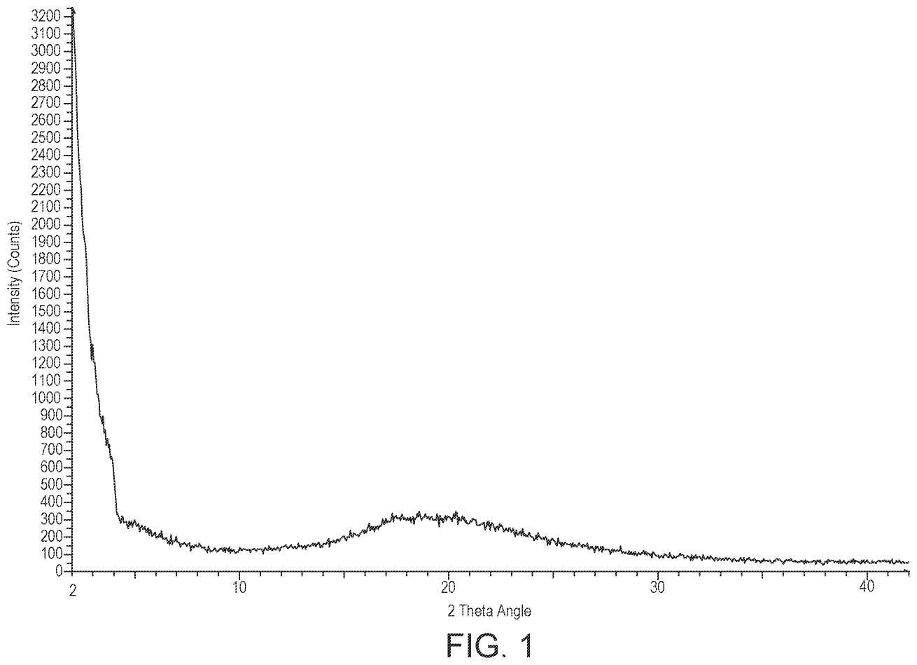

In one aspect, the present invention provides amorphous avapritinib. In one embodiment, the amorphous form may have the X-ray powder diffraction pattern substantially as shown in FIG. 1, in which it can be seen that the form has no long-range order in its lattice.

Amorphous avapritinib may be prepared by a process comprising the steps of:
(a) dissolving avapritinib in a suitable solvent to form a solution of avapritinib;
(b) flash cooling the solution of avapritinib; and
(c) quickly removing the solvent to form the amorphous avapritinib.

The solvent may be any suitable solvent which is capable of producing a solution of avapritinib. An example of a suitable solvent includes but is not limited to dioxane (e.g. 1,4-dioxane). The w/v ratio of avapritinib to solvent may be in the range of about 1 mg of avapritinib:about 1 to about 1000 μl of solvent, such as about 1 mg of avapritinib:about 1 to about 500 μl of solvent, for example about 1 mg of avapritinib:about 1 to about 100 μl of solvent, e.g. about 1 mg of avapritinib:about 30 μl of solvent.

The avapritinib may be dissolved in the solvent at ambient temperature or less. Alternatively, the avapritinib may be dissolved in the solvent at a temperature greater than ambient i.e. greater than 30° C. and below the boiling point of the reaction mixture. The boiling point of the reaction mixture may vary depending on the pressure under which the contacting step is conducted. In one embodiment, the dissolution step is carried out at atmospheric pressure (i.e. 1.0135×10⁵ Pa).

Flash cooling may occur by any suitable means, such as use of a dry ice/acetone bath.

The solvent may be removed by means of lyphilisation (i.e. solvent freeze-drying), spray drying, etc. The solvent is removed at a speed which does not allow the avapritinib to crystallise.

In another aspect, the present invention relates to a pharmaceutical composition comprising amorphous avapritinib as described herein and a pharmaceutically acceptable excipient.

In another aspect, the present invention relates to a method for treating cancer in a patient comprising administering a therapeutically effective amount of amorphous avapritinib as described herein to the patient. The method of treatment includes the treatment of gastrointestinal stromal tumours.

In another aspect, the present invention relates to amorphous avapritinib as described herein for use in treating cancer, such as the treatment of gastrointestinal stromal tumours.

In another aspect, the present invention relates to a method for treating mastocytosis in a patient comprising administering a therapeutically effective amount of amorphous avapritinib as described herein to the patient.

In another aspect, the present invention relates to amorphous avapritinib as described herein for use in treating mastocytosis.

### Avapritinib Anhydrate

It has been discovered that avapritinib can be prepared in a well-defined and consistently reproducible anhydrous crystalline form. Moreover, a reliable and scalable method for producing this anhydrous crystalline form has been developed. The avapritinib polymorph provided by the present invention is useful as an active ingredient in pharmaceutical formulations. In certain embodiments, the anhydrous crystalline form is purifiable. In certain embodiments and depending on time, temperature and humidity, the anhydrous crystalline form is stable. In certain embodiments, the anhydrous crystalline form is easy to isolate and handle. In certain embodiments, the process for preparing the anhydrous crystalline form is scalable.

The crystalline form described herein may be characterised using a number of methods known to the skilled person in the art, including single crystal X-ray diffraction, X-ray powder diffraction (XRPD), differential scanning calorimetry (DSC), thermal gravimetric analysis (TGA), infrared spectroscopy, Raman spectroscopy, nuclear magnetic resonance (NMR) spectroscopy (including solution and solid-state NMR). The chemical purity may be determined by standard analytical methods, such as thin layer chromatography (TLC), gas chromatography, high performance liquid chromatography (HPLC), and mass spectrometry (MS).

In one aspect, the present invention provides a crystalline form of avapritinib which is crystalline avapritinib anhydrate.

The crystalline avapritinib anhydrate may be free or substantially free of other polymorphic forms of avapritinib. In certain embodiments, the polymorphic purity of the anhydrate is ≥90%, ≥91%, ≥92%, ≥93%, ≥94%, ≥95% or higher. In certain embodiments, the polymorphic purity of the anhydrate is ≥95%. In certain embodiments, the polymorphic purity of the anhydrate is ≥96%. In certain embodiments, the polymorphic purity of the anhydrate is ≥97%. In certain embodiments, the polymorphic purity of the anhydrate is ≥98%. In certain embodiments, the polymorphic purity of the anhydrate is ≥99%.

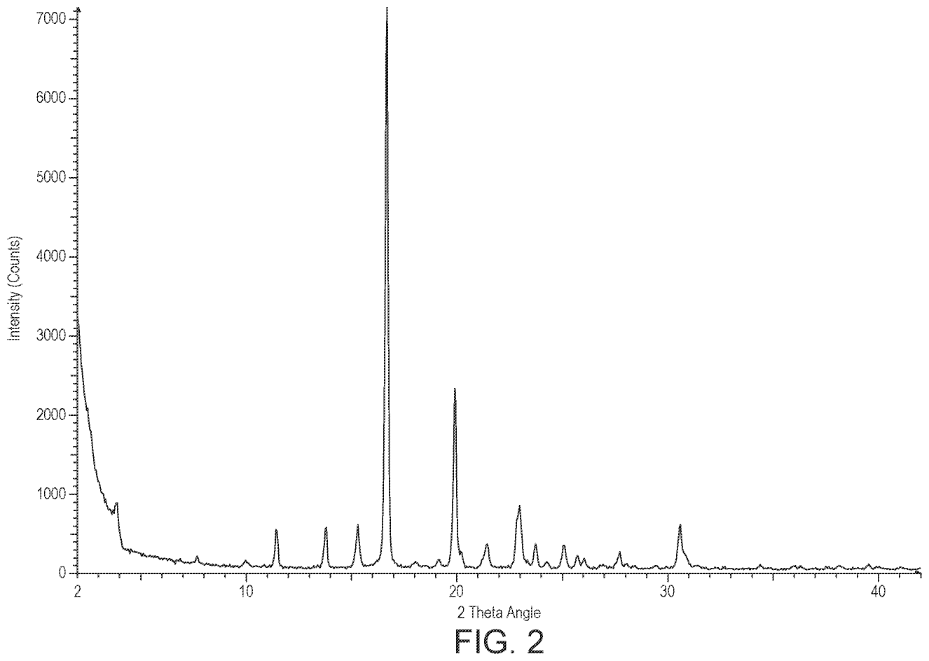

The anhydrate may have an X-ray powder diffraction pattern comprising one or more peaks (for example 1, 2, 3, 4, 5, 6, 7, 8, 9, or 10 peaks) selected from the group consisting of about 3.8, 7.6, 10.0, 11.5, 13.8, 15.3, 16.7, 18.0, 19.1, 19.9, 20.2, 21.4, 22.9, 23.7, 25.1, 25.7, 26.0, 27.7, and 30.6 degrees two-theta±0.2 degrees two-theta. In one embodiment, the anhydrate may have the X-ray powder diffraction pattern substantially as shown in FIG. 2.

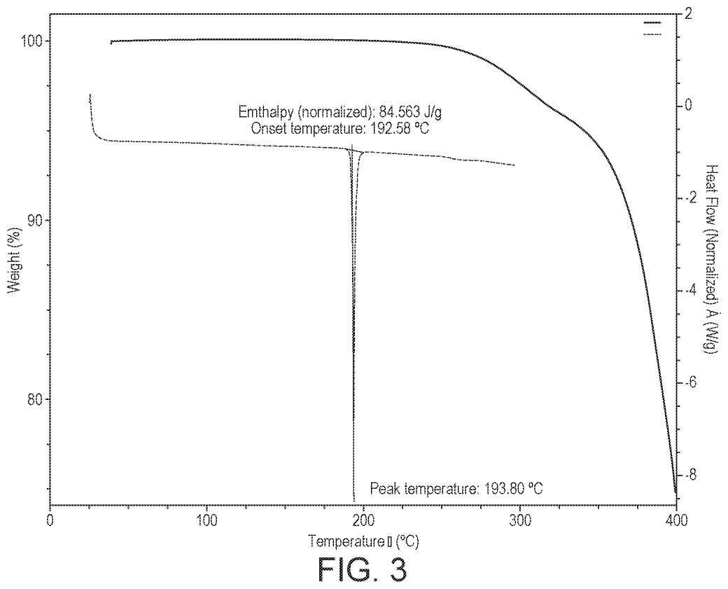

The anhydrate may have a DSC thermogram comprising an endothermic event with an onset at about 192.6° C. In one embodiment, the anhydrate may have a DSC thermogram substantially as shown in FIG. 3.

The anhydrate may have a TGA thermogram comprising no substantially mass loss when heated from about ambient temperature to about 200° C. In one embodiment, the anhydrate may have a TGA thermogram substantially as shown in FIG. 3.

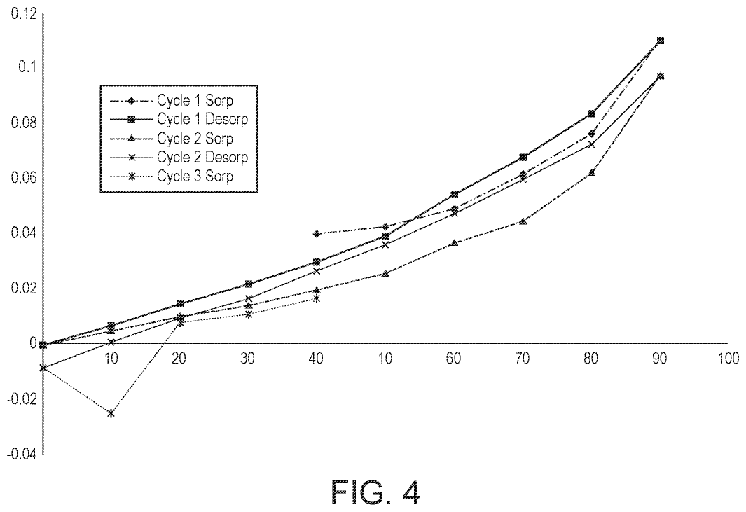

The anhydrate may have a GVS isotherm plot substantially as shown in FIG. 4. The GVS isotherm plot shows that the avapritinib anhydrate was characterised by a water uptake of about 0.1% w/w uptake at 25° C./90% RH. The XRPD after analysis showed that the crystalline avapritinib anhydrate was unchanged i.e. there was no change in form after GVS.

Crystalline avapritinib anhydrate may be prepared by a process comprising the steps of:
(a) contacting avapritinib with a solvent selected from the group consisting of acetone, dimethyl sulfoxide, ethyl acetate, methyl ethyl ketone, 2-methyl tetrahydrofuran, dichloromethane, nitromethane, 1,2-dimethyoxyethane, water, tert-butyl methyl ether, ethanol, heptane, isopropyl acetate, methyl isobutyl ketone, isopropanol, acetonitrile, toluene, 2-methyl-1-propanol, 1-propanol, ethanol, dimethylformamide, 1-methyl-2-pyrrolidinone, 2-methoxyethanol, and combinations thereof;
(b) forming a solution or suspension of avapritinib in the solvent; and
(c) recovering avapritinib anhydrate as a crystalline solid.

In one embodiment, the avapritinib which is contacted with a solvent is amorphous avapritinib. The amorphous avapritinib may be prepared by the method described herein.

In one embodiment, the solvent is a combination of acetone and water. The v/v ratio of acetone:water may be about 90:about 10 or about 95:about 5.

In another embodiment, the solvent is a combination of ethanol and water. The v/v ratio of ethanol:water may be about 95:about 5.

The quantity of solvent is not particularly limiting provided there is enough solvent to dissolve the avapritinib and form a solution, or suspend the avapritinib. The w/v ratio of avapritinib to solvent may be in the range of about 1 mg of avapritinib:about 1 to about 1000 μl of solvent, such as about 1 mg of avapritinib:about 1 to about 500 μl of solvent, for example about 1 mg of avapritinib:about 1 to about 150 μl of solvent, e.g. about 1 mg of avapritinib:about 5 to about 100 μl of solvent.

The avapritinib may be contacted with the solvent at ambient temperature or less. In one embodiment, the contacting step may be carried out at one or more temperatures in the range of ≥ about 0° C. to about ≤25° C. In some embodiments, the contacting step is carried out at one or more temperatures ≥ about 1° C. In some embodiments, the contacting step is carried out at one or more temperatures ≥ about 2° C. In some embodiments, the contacting step is carried out at one or more temperatures ≥ about 3° C. In some embodiments, the contacting step is carried out at one or more temperatures ≥ about 4° C. In some embodiments, the contacting step is carried out at one or more temperatures ≥ about 5° C. In some embodiments, the contacting step is carried out at one or more temperatures ≤ about 20° C. In some embodiments, the contacting step is carried out at one or more temperatures ≤ about 15° C. In some embodiments, the contacting step is carried out at one or more temperatures ≤ about 10° C. In one embodiment, the contacting step is carried out at one or more temperatures in the range of ≥ about 0° C. to ≤about 10° C., for example, about 5° C. In one embodiment, the contacting step may be carried out at ambient temperature e.g. about 25° C.

Alternatively, the avapritinib may be contacted with the solvent at a temperature greater than ambient i.e. greater than 30° C. and below the boiling point of the reaction mixture. The boiling point of the reaction mixture may vary depending on the pressure under which the contacting step is conducted. In one embodiment, the contacting step is carried out at atmospheric pressure (i.e. 1.0135×10⁵ Pa). In one embodiment, the contacting step may be carried out at one or more temperatures in the range of ≥ about 40° C. to about ≤60° C. In some embodiments, the contacting step is carried out at one or more temperatures ≥ about 41° C. In some embodiments, the contacting step is carried out at one or more temperatures ≥ about 42° C. In some embodiments, the contacting step is carried out at one or more temperatures ≥ about 43° C. In some embodiments, the contacting step is carried out at one or more temperatures ≥ about 44° C. In some embodiments, the contacting step is carried out at one or more temperatures ≥ about 45° C. In some embodiments, the contacting step is carried out at one or more temperatures ≥ about 46° C. In some embodiments, the contacting step is carried out at one or more temperatures ≥ about 47° C. In some embodiments, the contacting step is carried out at one or more temperatures ≥ about 48° C. In some embodiments, the contacting step is carried out at one or more temperatures ≥ about 49° C. In some embodiments, the contacting step is carried out at one or more temperatures ≥ about 50° C. In some embodiments, the contacting step is carried out at one or more temperatures ≤ about 59° C. In some embodiments, the contacting step is carried out at one or more temperatures ≤ about 58° C. In some embodiments, the contacting step is carried out at one or more temperatures ≤ about 57° C. In some embodiments, the contacting step is carried out at one or more temperatures ≤ about 56° C. In some embodiments, the contacting step is carried out at one or more temperatures ≤ about 55° C. In some embodiments, the contacting step is carried out at one or more temperatures ≤ about 54° C. In some embodiments, the contacting step is carried out at one or more temperatures ≤ about 53° C. In some embodiments, the contacting step is carried out at one or more temperatures ≤ about 52° C. In some embodiments, the contacting step is carried out at one or more temperatures ≤ about 51° C. In one embodiment, the contacting step is carried out at one or more temperatures in the range of ≥ about 45° C. to ≤ about 55° C. In one embodiment, the contacting step is carried out at a temperature of about 50° C.

The dissolution or suspension of avapritinib may be encouraged through the use of an aid such as stirring, shaking and/or sonication. Additional solvent may be added to aid the dissolution or suspension of the avapritinib.

The solution or suspension may then be cooled such that the resulting solution or suspension has a temperature below that of the solution or suspension of step (b). The rate of cooling may be from about 0.05° C./minute to about 2° C./minute, such as about 0.1° C./minute to about 1.5° C./minute, for example about 0.1° C./minute. When a solution of avapritinib is cooled, a suspension may eventually be observed. When a suspension of avapritinib is cooled, no perceptible change in the appearance of the suspension may occur.

The solution or suspension may be cooled to ambient temperature or a temperature of less than ambient temperature. In one embodiment, the solution or suspension may be cooled to one or more temperatures in the range of ≥ about 0° C. to about ≤20° C. In some embodiments, the solution or suspension is cooled to one or more temperatures ≥ about 1° C. In some embodiments, the solution or suspension is cooled to one or more temperatures ≥ about 2° C. In some embodiments, the solution or suspension is cooled to one or more temperatures ≥ about 3° C. In some embodiments, the solution or suspension is cooled to one or more temperatures ≥ about 4° C. In some embodiments, the solution or suspension is cooled to one or more temperatures ≥ about 5° C. In some embodiments, the solution or suspension is cooled to one or more temperatures ≤ about 15° C. In some embodiments, the solution or suspension is cooled to one or more temperatures ≤ about 14° C. In some embodiments, the solution or suspension is cooled to one or more temperatures ≤ about 13° C. In some embodiments, the solution or suspension is cooled to one or more temperatures ≤ about 12° C. In some embodiments, the solution or suspension may be cooled to one or more temperatures ≤ about 11° C. In some embodiments, the solution or suspension is cooled to one or more temperatures ≤ about 10° C. In one embodiment, the solution or suspension is cooled to one or more temperatures in the range of about 5° C. to about 10° C.

The reaction mixture may then be stirred, shaken and/or sonicated for a further period of time.

In step (c), the avapritinib anhydrate is recovered as a crystalline solid. The crystalline anhydrate may be recovered by directly by filtering, decanting or centrifuging. If desired, the suspension may be mobilised with additional portions of the solvent prior to recovery of the crystalline solid. Alternatively, a proportion or substantially all of the solvent may be evaporated prior to recovery of the crystalline solid.

Howsoever the crystalline anhydrate is recovered, the separated anhydrate may be washed with solvent (e.g. one or more of the solvents described above) and dried. Drying may be performed using known methods, for example, at temperatures in the range of about 10° C. to about 60° C., such as about 20° C. to about 40° C., for example, ambient temperature under vacuum (for example about 1 mbar to about 30 mbar) for about 1 hour to about 24 hours. It is preferred that the drying conditions are maintained below the point at which the anhydrate degrades and so when the anhydrate is known to degrade within the temperature or pressure ranges given above, the drying conditions should be maintained below the degradation temperature or vacuum.

Steps (a) to (c) may be carried out one or more times (e.g. 1, 2, 3, 4 or 5 times). When steps (a) to (c) are carried out more than once (e.g. 2, 3, 4 or 5 times), step (a) may be optionally seeded with crystalline avapritinib anhydrate which was previously prepared and isolated by the first iteration of steps (a) to (c).

Alternatively or in addition, when steps (a) to (c) are carried out more than once (e.g. 2, 3, 4 or 5 times), the solution or suspension formed in step (b) may be optionally seeded with crystalline avapritinib anhydrate (which was previously prepared and isolated by a method described herein).

The crystalline avapritinib anhydrate formed may be free or substantially free of other polymorphic forms of avapritinib. In certain embodiments, the polymorphic purity of the anhydrate is ≥90%, ≥91%, ≥92%, ≥93%, ≥94%, ≥95% or higher. In certain embodiments, the polymorphic purity of the anhydrate is ≥95%. In certain embodiments, the polymorphic purity of the anhydrate is ≥96%. In certain embodiments, the polymorphic purity of the anhydrate is ≥97%. In certain embodiments, the polymorphic purity of the anhydrate is ≥98%. In certain embodiments, the polymorphic purity of the anhydrate is ≥99%.

In another aspect, crystalline avapritinib anhydrate may be prepared by a process comprising the steps of:
(a) dissolving avapritinib in a first solvent selected from the group consisting of dimethyl sulfoxide, dimethylformamide, dichloromethane, dioxane (e.g. 1,4-dioxane), tetrahydrofuran, and combinations thereof;
(b) adding a second solvent to form a suspension of avapritinib, wherein the second solvent is selected from the group consisting of tert-butyl methyl ether, ethanol, isopropyl acetate, water, acetonitrile, toluene, heptane (e.g. n-heptane), and combinations thereof; and
(c) recovering avapritinib anhydrate as a crystalline solid.

In one embodiment, the avapritinib which is contacted with a solvent is amorphous avapritinib. The amorphous avapritinib may be prepared by the method described herein.

The quantity of the first solvent is not particularly limiting provided there is enough solvent to dissolve the avapritinib and form a solution, or suspend the avapritinib. The w/v ratio of avapritinib to the first solvent may be in the range of about 1 mg of avapritinib:about 1 to about 100 μl of solvent, such as about 1 mg of avapritinib:about 1 to about 50 μl of solvent, for example about 1 mg of avapritinib:about 1 to about 20 μl of solvent, e.g. about 1 mg of avapritinib:about 10 to about 10 μl of solvent.

The avapritinib may be dissolved in the first solvent at ambient temperature or less. In one embodiment, the dissolving step may be carried out at one or more temperatures in the range of ≥ about 0° C. to about ≤25° C. In some embodiments, the dissolving step is carried out at one or more temperatures ≥ about 1° C. In some embodiments, the dissolving step is carried out at one or more temperatures ≥ about 2° C. In some embodiments, the dissolving step is carried out at one or more temperatures ≥ about 3° C. In some embodiments, the dissolving step is carried out at one or more temperatures ≥ about 4° C. In some embodiments, the dissolving step is carried out at one or more temperatures ≥ about 5° C. In some embodiments, the dissolving step is carried out at one or more temperatures ≥ about 20° C. In some embodiments, the dissolving step is carried out at one or more temperatures ≥ about 15° C. In some embodiments, the dissolving step is carried out at one or more temperatures ≤ about 10° C. In one embodiment, the dissolving step is carried out at one or more temperatures in the range of ≥ about 0° C. to about 10° C., for example, about 5° C. In one embodiment, the dissolving step may be carried out at about ambient temperature e.g. about 25° C.

Alternatively, the avapritinib may be dissolved in the solvent at a temperature greater than ambient i.e. greater than 30° C. and below the boiling point of the reaction mixture. The boiling point of the reaction mixture may vary depending on the pressure under which the contacting step is conducted. In one embodiment, the dissolving step is carried out at atmospheric pressure (i.e. 1.0135×10⁵ Pa). In one embodiment, the dissolving step may be carried out at one or more temperatures in the range of ≥ about 40° C. to about ≤60° C. In some embodiments, the dissolving step is carried out at one or more temperatures ≥ about 41° C. In some embodiments, the dissolving step is carried out at one or more temperatures ≥ about 42° C. In some embodiments, the dissolving step is carried out at one or more temperatures ≥ about 43° C. In some embodiments, the dissolving step is carried out at one or more temperatures ≥ about 44° C. In some embodiments, the dissolving step is carried out at one or more temperatures ≥ about 45° C. In some embodiments, the dissolving step is carried out at one or more temperatures ≥ about 46° C. In some embodiments, the dissolving step is carried out at one or more temperatures ≥ about 47° C. In some embodiments, the dissolving step is carried out at one or more temperatures ≥ about 48° C. In some embodiments, the dissolving step is carried out at one or more temperatures ≥ about 49° C. In some embodiments, the dissolving step is carried out at one or more temperatures ≥ about 50° C. In some embodiments, the dissolving step is carried out at one or more temperatures ≤ about 59° C. In some embodiments, the dissolving step is carried out at one or more temperatures ≤ about 58° C. In some embodiments, the dissolving step is carried out at one or more temperatures ≤ about 57° C. In some embodiments, the dissolving step is carried out at one or more temperatures ≤ about 56° C. In some embodiments, the dissolving step is carried out at one or more temperatures ≤ about 55° C. In some embodiments, the dissolving step is carried out at one or more temperatures ≤ about 54° C. In some embodiments, the dissolving step is carried out at one or more temperatures ≤ about 53° C. In some embodiments, the dissolving step is carried out at one or more temperatures ≤ about 52° C. In some embodiments, the dissolving step is carried out at one or more temperatures ≤ about 51° C. In one embodiment, the dissolving step is carried out at one or more temperatures in the range of ≥ about 45° C. to ≤ about 55° C. In one embodiment, the dissolving step is carried out at a temperature of about 50° C.

The dissolution of avapritinib may be encouraged through the use of an aid such as stirring, shaking and/or sonication. Additional solvent may be added to aid the dissolution of the avapritinib.

In step (b), a second solvent is added to the reaction mixture to form a suspension of avapritinib, wherein the second solvent is selected from the group consisting of tert-butyl methyl ether, ethanol, isopropyl acetate, water, acetonitrile, toluene, heptane (e.g. n-heptane), and combinations thereof.

The quantity of the second solvent is not particularly limiting provided there is enough solvent to form a suspension the avapritinib in the solvent mixture. The w/v ratio of avapritinib to the second solvent may be in the range of about 1 mg of avapritinib:about 1 to about 200 μl of solvent, such as about 1 mg of avapritinib:about 1 to about 150 μl of solvent, for example about 1 mg of avapritinib:about 1 to about 100 μl of solvent, e.g. about 1 mg of avapritinib:about 5 to about 50 μl of solvent. These w/v ratios have been calculated using the mass of avapritinib initially dissolved in the first solvent i.e. the quantity of avapritinib inputted into the process.

After the addition of the second solvent, the reaction mixture may be treated at ambient temperature or less as described above in connection with first solvent.

Alternatively, the avapritinib may be dissolved in the solvent at a temperature greater than ambient i.e. greater than 30° C. and below the boiling point of the reaction mixture as described above in connection with the first solvent.

In one embodiment, the first solvent is dimethyl sulfoxide and the second solvent is selected from the group consisting of tert-butyl methyl ether, ethanol, isopropyl acetate, water, acetonitrile, and combinations thereof.

In another embodiment, the first solvent is dimethylformamide and the second solvent is selected from the group consisting of tert-butyl methyl ether, ethanol, isopropyl acetate, water, acetonitrile, toluene, and combinations thereof.

In another embodiment, the first solvent is dichloromethane and the second solvent is selected from the group consisting of tert-butyl methyl ether, ethanol, isopropyl acetate, acetonitrile, toluene, heptane (e.g. n-heptane), and combinations thereof.

In another embodiment, the first solvent is dioxane (e.g. 1,4-dioxane) and the second solvent is selected from the group consisting of tert-butyl methyl ether, ethanol, isopropyl acetate, water, acetonitrile, toluene, and combinations thereof.

In another embodiment, the first solvent is tetrahydrofuran and the second solvent is selected from the group consisting of tert-butyl methyl ether, ethanol, isopropyl acetate, water, acetonitrile, toluene, heptane (e.g. n-heptane), and combinations thereof.

In one embodiment, the first solvent is tetrahydrofuran and the second solvent is heptane (e.g. n-heptane).

The reaction mixture may then be stirred, shaken and/or sonicated for a further period of time.

The solution or suspension may then be cooled such that the resulting solution or suspension has a temperature below that of the solution or suspension of step (b). The rate of cooling may be from about 0.05° C./minute to about 2° C./minute, such as about 0.1° C./minute to about 1.5° C./minute, for example about 0.1° C./minute. When a solution of avapritinib is cooled, a suspension may eventually be observed. When a suspension of avapritinib is cooled, no perceptible change in the appearance of the suspension may occur.

The solution or suspension may be cooled to ambient temperature or a temperature of less than ambient temperature. In one embodiment, the solution or suspension may be cooled to one or more temperatures in the range of ≥ about 0° C. to about ≤20° C. In some embodiments, the solution or suspension is cooled to one or more temperatures ≥ about 1° C. In some embodiments, the solution or suspension is cooled to one or more temperatures ≥ about 2° C. In some embodiments, the solution or suspension is cooled to one or more temperatures ≥ about 3° C. In some embodiments, the solution or suspension is cooled to one or more temperatures ≥ about 4° C. In some embodiments, the solution or suspension is cooled to one or more temperatures ≥ about 5° C. In some embodiments, the solution or suspension is cooled to one or more temperatures ≤ about 15° C. In some embodiments, the solution or suspension is cooled to one or more temperatures ≤ about 14° C. In some embodiments, the solution or suspension is cooled to one or more temperatures ≤ about 13° C. In some embodiments, the solution or suspension is cooled to one or more temperatures ≤ about 12° C. In some embodiments, the solution or suspension may be cooled to one or more temperatures ≤ about 11° C. In some embodiments, the solution or suspension is cooled to one or more temperatures ≤ about 10° C. In one embodiment, the solution or suspension is cooled to one or more temperatures in the range of about 5° C. to about 10° C.

In step (c), the avapritinib anhydrate is recovered as a crystalline solid. The crystalline anhydrate may be recovered by directly by filtering, decanting or centrifuging. If desired, the suspension may be mobilised with additional portions of the solvent prior to recovery of the crystalline solid. Alternatively, a proportion or substantially all of the solvent may be evaporated prior to recovery of the crystalline solid.

Howsoever the crystalline anhydrate is recovered, the separated anhydrate may be washed with solvent (e.g. one or more of the solvents described above) and dried. Drying may be performed using known methods, for example, at temperatures in the range of about 10° C. to about 60° C., such as about 20° C. to about 40° C., for example, ambient temperature under vacuum (for example about 1 mbar to about 30 mbar) for about 1 hour to about 24 hours. It is preferred that the drying conditions are maintained below the point at which the anhydrate degrades and so when the anhydrate is known to degrade within the temperature or pressure ranges given above, the drying conditions should be maintained below the degradation temperature or vacuum.

Steps (a) to (c) may be carried out one or more times (e.g. 1, 2, 3, 4 or 5 times). When steps (a) to (c) are carried out more than once (e.g. 2, 3, 4 or 5 times), step (a) may be optionally seeded with crystalline avapritinib anhydrate which was previously prepared and isolated by a method described herein).

Alternatively or in addition, when steps (a) to (c) are carried out more than once (e.g. 2, 3, 4 or 5 times), the solution or suspension formed in step (b) may be optionally seeded with crystalline avapritinib anhydrate (which was previously prepared and isolated by a method described herein).

The crystalline avapritinib anhydrate formed may be free or substantially free of other polymorphic forms of avapritinib. In certain embodiments, the polymorphic purity of the anhydrate is ≥90%, ≥91%, ≥92%, ≥93%, ≥94%, ≥95% or higher. In certain embodiments, the polymorphic purity of the anhydrate is ≥95%. In certain embodiments, the polymorphic purity of the anhydrate is ≥96%. In certain embodiments, the polymorphic purity of the anhydrate is ≥97%. In certain embodiments, the polymorphic purity of the anhydrate is ≥98%. In certain embodiments, the polymorphic purity of the anhydrate is ≥99%.

In another aspect, the present invention relates to a pharmaceutical composition comprising crystalline avapritinib anhydrate as described herein and a pharmaceutically acceptable excipient.

In another aspect, the present invention relates to a method for treating cancer in a patient comprising administering a therapeutically effective amount of crystalline avapritinib anhydrate as described herein to the patient. The method of treatment includes the treatment of gastrointestinal stromal tumours.

In another aspect, the present invention relates to crystalline avapritinib anhydrate as described herein for use in treating cancer, such as the treatment of gastrointestinal stromal tumours.

In another aspect, the present invention relates to a method for treating mastocytosis in a patient comprising administering a therapeutically effective amount of crystalline avapritinib anhydrate as described herein to the patient.

In another aspect, the present invention relates to crystalline avapritinib anhydrate as described herein for use in treating mastocytosis.

### Avapritinib Methanol Solvate

It has been discovered that avapritinib can be prepared in a well-defined and consistently reproducible methanol solvate form. Moreover, a reliable and scalable method for producing this solvate form has been developed. The avapritinib polymorph provided by the present invention is useful as an active ingredient in pharmaceutical formulations. In certain embodiments, the crystalline solvate form is purifiable. In certain embodiments and depending on time, temperature and humidity, the crystalline solvate form is stable. In certain embodiments, the crystalline solvate form is easy to isolate and handle. In certain embodiments, the process for preparing the crystalline solvate form is scalable.

The crystalline form described herein may be characterised using a number of methods known to the skilled person in the art, including single crystal X-ray diffraction, X-ray powder diffraction (XRPD), differential scanning calorimetry (DSC), thermal gravimetric analysis (TGA), infrared spectroscopy, Raman spectroscopy, nuclear magnetic resonance (NMR) spectroscopy (including solution and solid-state NMR). The chemical purity may be determined by standard analytical methods, such as thin layer chromatography (TLC), gas chromatography, high performance liquid chromatography (HPLC), and mass spectrometry (MS).

In one aspect, the present invention provides a crystalline form of avapritinib which is crystalline avapritinib methanol solvate.

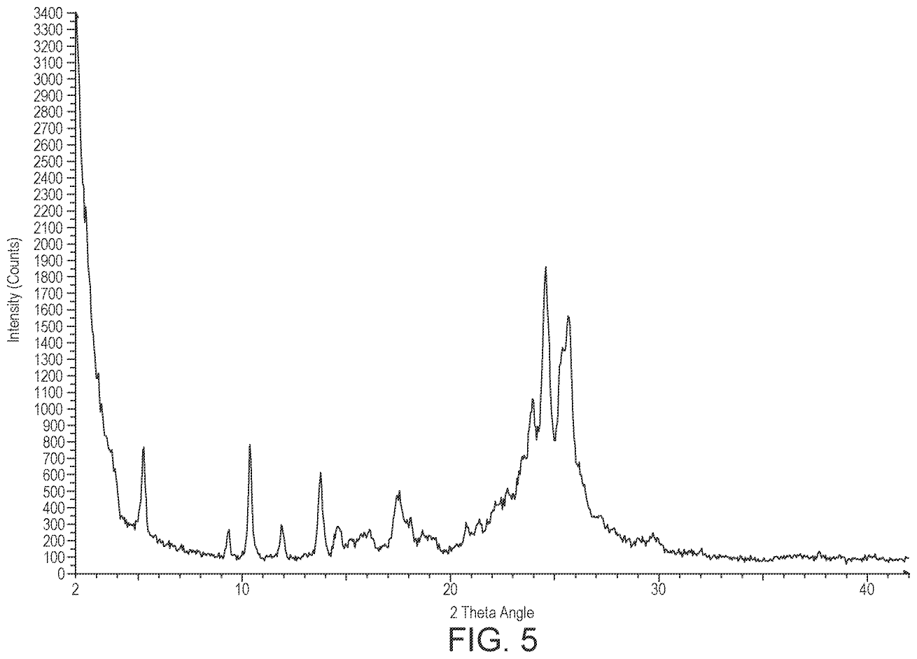

The methanol solvate may have an X-ray powder diffraction pattern comprising one or more peaks (for example 1, 2, 3, 4, 5, 6, 7, 8, 9, or 10 peaks) selected from the group consisting of about 5.2, 9.3, 10.4, 11.9, 13.7, 14.6, 16.1, 17.5, 18.7, 20.8, 21.4, 23.9, 24.6, 25.4, and 25.7 degrees two-theta±0.2 degrees two-theta. In one embodiment, the anhydrate may have the X-ray powder diffraction pattern substantially as shown in FIG. 5.

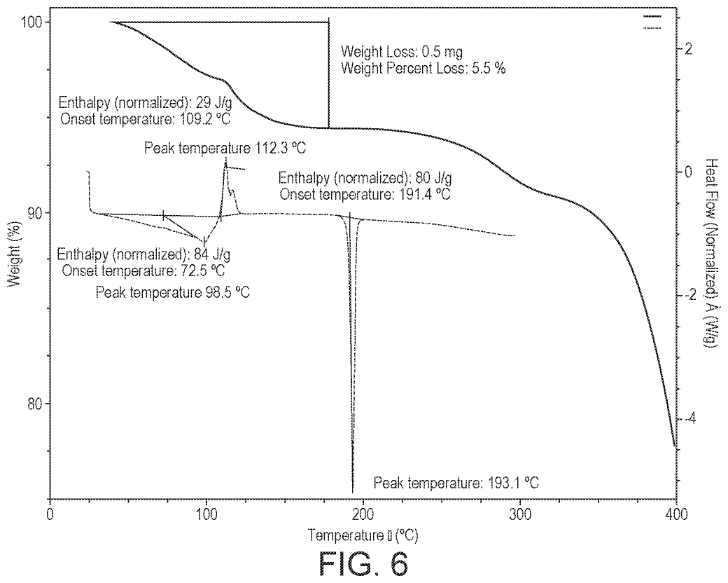

The methanol solvate may have a DSC thermogram comprising two endothermic events with onset temperatures of about 72.5° C. and about 191.4° C. The DSC thermogram of the methanol solvate may also comprise an exothermic event with an onset temperature at about 109.2° C. As the exothermic event is a kinetic event, the skilled person would understand that the onset is variable and may occur at different temperatures depending on the conditions under which the sample is analysed, for example, the DSC instrument used to analyse the sample. In one embodiment, the anhydrate may have a DSC thermogram substantially as shown in FIG. 6.

The methanol solvate may have a TGA thermogram comprising about 5.5% mass loss when heated from about ambient temperature to about 200° C. In one embodiment, the anhydrate may have a TGA thermogram substantially as shown in FIG. 6.

The crystalline avapritinib methanol solvate formed may be free or substantially free of other polymorphic forms of avapritinib. In certain embodiments, the polymorphic purity of the solvate is ≥90%, ≥91%, ≥92%, ≥93%, ≥94%, ≥95% or higher. In certain embodiments, the polymorphic purity of the solvate is ≥95%. In certain embodiments, the polymorphic purity of the solvate is ≥96%. In certain embodiments, the polymorphic purity of the solvate is ≥97%. In certain embodiments, the polymorphic purity of the solvate is ≥98%. In certain embodiments, the polymorphic purity of the solvate is ≥99%.

Avapritinib methanol solvate may be prepared by a process comprising the steps of:
(a) contacting avapritinib with methanol; and
(b) forming a suspension of avapritinib in methanol.

The quantity of methanol is not particularly limiting provided there is enough methanol to substantially suspend the avapritinib. The w/v ratio of avapritinib to methanol solvent may be in the range of about 1 mg of avapritinib:about 1 to about 1000 μl of methanol, such as about 1 mg of avapritinib:about 1 to about 500 μl of methanol, for example about 1 mg of avapritinib:about 1 to about 250 μl of methanol, e.g. about 1 mg of avapritinib:about 5 to about 100 μl of methanol.

The avapritinib may be contacted with methanol at ambient temperature or less. In one embodiment, the contacting step may be carried out at one or more temperatures in the range of ≥ about 0° C. to about ≤25° C. In some embodiments, the contacting step is carried out at one or more temperatures ≥ about 1° C. In some embodiments, the contacting step is carried out at one or more temperatures ≥ about 2° C. In some embodiments, the contacting step is carried out at one or more temperatures ≥ about 3° C. In some embodiments, the contacting step is carried out at one or more temperatures ≥ about 4° C. In some embodiments, the contacting step is carried out at one or more temperatures ≥ about 5° C. In some embodiments, the contacting step is carried out at one or more temperatures ≤ about 20° C. In some embodiments, the contacting step is carried out at one or more temperatures ≤ about 15° C. In some embodiments, the contacting step is carried out at one or more temperatures ≤ about 10° C. In one embodiment, the contacting step is carried out at one or more temperatures in the range of ≥ about 0° C. to ≤ about 10° C., for example, about 5° C. In one embodiment, the contacting step may be carried out at ambient temperature e.g. about 25° C.

Alternatively, the avapritinib may be contacted with methanol at a temperature greater than ambient i.e. greater than 30° C. and below the boiling point of the reaction mixture. The boiling point of the reaction mixture may vary depending on the pressure under which the contacting step is conducted. Methanol has a boiling point of about 64.7° C. at atmospheric pressure (i.e. 1.0135×10⁵ Pa). In one embodiment, the contacting step may be carried out at one or more temperatures in the range of ≥ about 30° C. to about <65° C. In some embodiments, the contacting step is carried out at one or more temperatures ≥ about 41° C. In some embodiments, the contacting step is carried out at one or more temperatures ≥ about 42° C. In some embodiments, the contacting step is carried out at one or more temperatures ≥ about 43° C. In some embodiments, the contacting step is carried out at one or more temperatures ≥ about 44° C. In some embodiments, the contacting step is carried out at one or more temperatures ≥ about 45° C. In some embodiments, the contacting step is carried out at one or more temperatures ≥ about 46° C. In some embodiments, the contacting step is carried out at one or more temperatures ≥ about 47° C. In some embodiments, the contacting step is carried out at one or more temperatures ≥ about 48° C. In some embodiments, the contacting step is carried out at one or more temperatures ≥ about 49° C. In some embodiments, the contacting step is carried out at one or more temperatures ≥ about 50° C. In some embodiments, the contacting step is carried out at one or more temperatures ≤ about 60° C. In some embodiments, the contacting step is carried out at one or more temperatures ≤ about 59° C. In some embodiments, the contacting step is carried out at one or more temperatures ≤ about 58° C. In some embodiments, the contacting step is carried out at one or more temperatures ≤ about 57° C. In some embodiments, the contacting step is carried out at one or more temperatures ≤ about 56° C. In some embodiments, the contacting step is carried out at one or more temperatures ≤ about 55° C. In some embodiments, the contacting step is carried out at one or more temperatures ≤ about 54° C. In some embodiments, the contacting step is carried out at one or more temperatures ≤ about 53° C. In some embodiments, the contacting step is carried out at one or more temperatures ≤ about 52 ° C. In some embodiments, the contacting step is carried out at one or more temperatures ≤ about 51° C. In one embodiment, the contacting step is carried out at one or more temperatures in the range of ≥ about 45° C. to ≤ about 55° C. In one embodiment, the contacting step is carried out at a temperature of about 50° C.

Alternatively, avapritinib may be contacted with methanol at ambient temperature or less and then matured between this temperature and one or more temperatures greater than ambient temperature. Ambient temperature, temperatures less than ambient, and temperatures greater than ambient are as described above. The maturation step may comprise oscillating the temperature for a period of time (e.g. about 4 hours) at ambient, a period of time at the temperature greater than ambient (e.g. about 4 hours), followed by another period of time (e.g. about 4 hours) at ambient, and so on, for an extended period of time (e.g. about 4 days).

The suspension of avapritinib may be agitated through the use of an aid such as stirring, shaking and/or sonication.

The process may further comprise the step of recovering avapritinib methanol solvate as a crystalline solid. The crystalline solvate may be recovered by directly by filtering, decanting or centrifuging. If desired, the suspension may be mobilised with additional portions of methanol prior to recovery of the crystalline solid. Alternatively, a proportion or substantially all of the methanol solvent may be evaporated prior to recovery of the crystalline solid.

Howsoever the crystalline methanol solvate is recovered, the separated solvate may be washed with alcohol and dried. Drying may be performed using known methods, for example, at temperatures in the range of about 10° C. to about 60° C., such as about 20° C. to about 40° C., for example, ambient temperature under vacuum (for example about 1 mbar to about 30 mbar) for about 1 hour to about 24 hours. It is preferred that the drying conditions are maintained below the point at which the methanol solvate desolvates and so when the solvate is known to desolvate within the temperature or pressure ranges given above, the drying conditions should be maintained below the desolvation temperature or vacuum.

### Avapritinib Hydrate

It has been discovered that avapritinib can be prepared in a well-defined and consistently reproducible hydrate form. Moreover, a reliable and scalable method for producing this hydrate form has been developed. The avapritinib polymorph provided by the present invention is useful as an active ingredient in pharmaceutical formulations. In certain embodiments, the crystalline hydrate form is purifiable. In certain embodiments and depending on time, temperature and humidity, the crystalline hydrate form is stable. In certain embodiments, the crystalline hydrate form is easy to isolate and handle. In certain embodiments, the process for preparing the crystalline hydrate form is scalable.

The crystalline form described herein may be characterised using a number of methods known to the skilled person in the art, including single crystal X-ray diffraction, X-ray powder diffraction (XRPD), differential scanning calorimetry (DSC), thermal gravimetric analysis (TGA), infrared spectroscopy, Raman spectroscopy, nuclear magnetic resonance (NMR) spectroscopy (including solution and solid-state NMR). The chemical purity may be determined by standard analytical methods, such as thin layer chromatography (TLC), gas chromatography, high performance liquid chromatography (HPLC), and mass spectrometry (MS).

In one aspect, the present invention provides a crystalline form of avapritinib which is crystalline avapritinib hydrate.

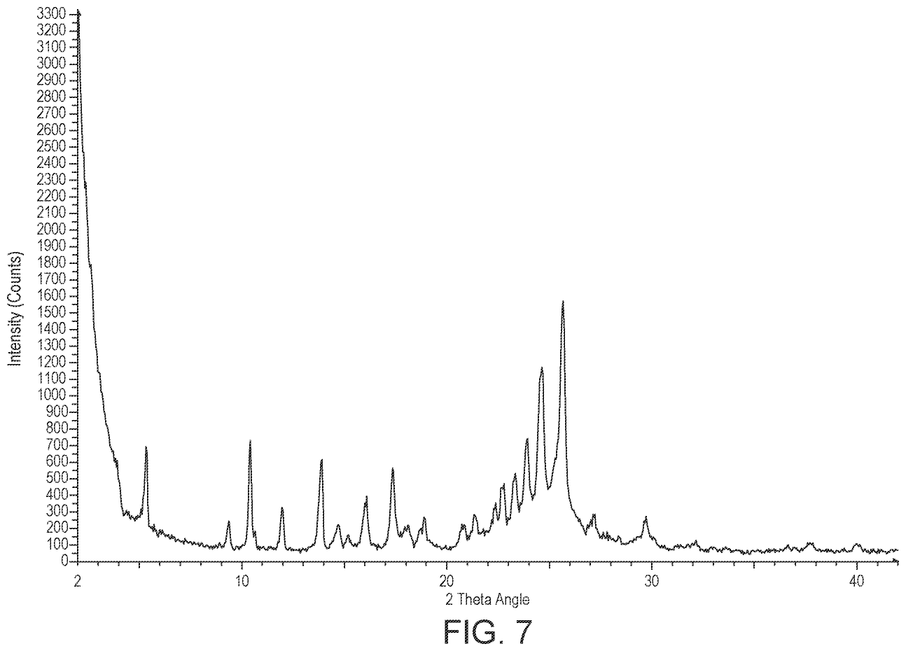

The hydrate may have an X-ray powder diffraction pattern comprising one or more peaks (for example 1, 2, 3, 4, 5, 6, 7, 8, 9, or 10 peaks) selected from the group consisting of about 5.3, 9.3, 10.4, 10.7, 12.0, 13.9, 14.7, 15.2, 16.1, 17.4, 17.9, 18.7, 18.9, 20.8, 21.4, 22.4, 22.7, 23.3, 23.9, 24.6, 25.6, 27.2, 28.4, and 29.7 degrees two-theta±0.2 degrees two-theta. In one embodiment, the anhydrate may have the X-ray powder diffraction pattern substantially as shown in FIG. 7.

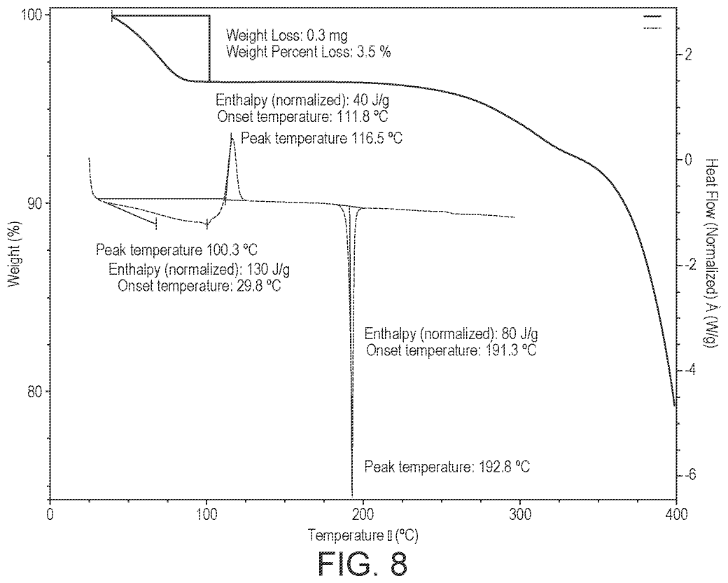

The hydrate may have a DSC thermogram comprising two endothermic events with onset temperatures of about 29.8° C. and about 191.3° C. The DSC thermogram of the hydrate may also comprise an exothermic event with an onset temperature at about 116.5° C. As the exothermic event is a kinetic event, the skilled person would understand that the onset is variable and may occur at different temperatures depending on the conditions under which the sample is analysed, for example, the DSC instrument used to analyse the sample. In one embodiment, the anhydrate may have a DSC thermogram substantially as shown in FIG. 8.

The hydrate may have a TGA thermogram comprising about 3.5% mass loss when heated from about ambient temperature to about 200° C. In one embodiment, the hydrate may have a TGA thermogram substantially as shown in FIG. 8.

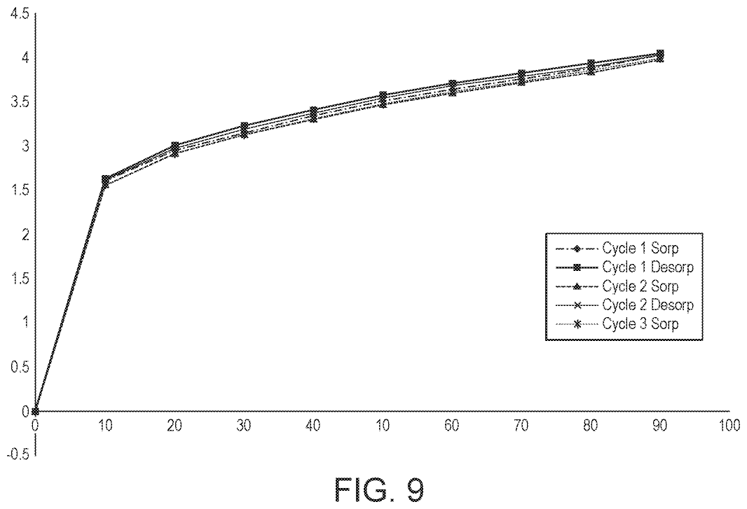

The hydrate may have a GVS isotherm plot substantially as shown in FIG. 9. The GVS isotherm plot shows that the avapritinib hydrate was characterised by a water uptake of about 4.0% w/w uptake at 25° C./90% RH. The XRPD after analysis showed that the crystalline avapritinib hydrate was unchanged i.e. there was no change in form after GVS.

The crystalline avapritinib hydrate formed may be free or substantially free of other polymorphic forms of avapritinib. In certain embodiments, the polymorphic purity of the hydrate is ≥90%, ≥91%, ≥92%, ≥93%, ≥94%, ≥95% or higher. In certain embodiments, the polymorphic purity of the hydrate is ≥95%. In certain embodiments, the polymorphic purity of the hydrate is ≥96%. In certain embodiments, the polymorphic purity of the hydrate is ≥97%. In certain embodiments, the polymorphic purity of the hydrate is ≥98%. In certain embodiments, the polymorphic purity of the hydrate is ≥99%.

Without wishing to be bound by theory, it is believed the hydrate of the invention is a channel hydrate. It is also believed the hydrate is isostructural to the crystalline avapritinib methanol solvate described herein.

Crystalline avapritinib hydrate may be prepared by a process comprising the step of hydrating crystalline avapritinib methanol solvate.

Hydration may be affected by exposing the methanol solvate to water, particularly water vapour. For example, the methanol solvate may be placed within an enclosed chamber under vacuum and water vapour (for instance, in the form of moist air or moist nitrogen with a relative humidity (RH) of e.g. about 75% or about 97%) may be bled into the enclosed chamber. Alternatively, the methanol solvate may be exposed to a moist atmosphere at approximately atmospheric pressure within an enclosed chamber containing a source of liquid water at a temperature from about ambient temperature to about 50° C., for example, about 25° C., or about 40° C.

Alternatively, hydration may be affected by slurrying the methanol solvate in water for a time (e.g. overnight) and at a temperature (e.g. ambient temperature) effective to form avapritinib hydrate.

Howsoever the crystalline hydrate is recovered, the separated hydrate may be washed with water and dried. Drying may be performed using known methods, for example, at temperatures in the range of about 10° C. to about 60° C., such as about 20° C. to about 40° C., for example, ambient temperature under vacuum (for example about 1 mbar to about 30 mbar) for about 1 hour to about 24 hours. It is preferred that the drying conditions are maintained below the point at which the hydrate degrades and so when the hydrate is known to degrade within the temperature or pressure ranges given above, the drying conditions should be maintained below the degradation temperature or vacuum.

The crystalline avapritinib hydrate formed may be free or substantially free of other polymorphic forms of avapritinib. In certain embodiments, the polymorphic purity of the hydrate is ≥90%, ≥91%, ≥92%, ≥93%, ≥94%, ≥95% or higher. In certain embodiments, the polymorphic purity of the hydrate is ≥95%. In certain embodiments, the polymorphic purity of the hydrate is ≥96%. In certain embodiments, the polymorphic purity of the hydrate is ≥97%. In certain embodiments, the polymorphic purity of the hydrate is ≥98%. In certain embodiments, the polymorphic purity of the hydrate is ≥99%.

In another aspect, the present invention relates to a pharmaceutical composition comprising crystalline avapritinib hydrate as described herein and a pharmaceutically acceptable excipient.

In another aspect, the present invention relates to a method for treating cancer in a patient comprising administering a therapeutically effective amount of crystalline avapritinib hydrate as described herein to the patient. The method of treatment includes the treatment of gastrointestinal stromal tumours.

In another aspect, the present invention relates to crystalline avapritinib hydrate as described herein for use in treating cancer, such as the treatment of gastrointestinal stromal tumours.

In another aspect, the present invention relates to a method for treating mastocytosis in a patient comprising administering a therapeutically effective amount of crystalline avapritinib hydrate as described herein to the patient.

In another aspect, the present invention relates to crystalline avapritinib hydrate as described herein for use in treating mastocytosis.

Embodiments and/or optional features of the invention have been described above. Any aspect of the invention may be combined with any other aspect of the invention, unless the context demands otherwise. Any of the embodiments or optional features of any aspect may be combined, singly or in combination, with any aspect of the invention, unless the context demands otherwise.

The invention will now be described further by reference to the following examples, which are intended to illustrate but not limit, the scope of the invention.

### EXAMPLES

### 1 Instrument and Methodology Details

### 1.1 X-Ray Powder Diffraction (XRPD)

### 1.1.1 Bruker AXS D8 Advance

XRPD diffractograms were collected on a Bruker D8 diffractometer using Cu Kα radiation (40 kV, 40 mA) and a θ-2θ goniometer fitted with a Ge monochromator. The incident beam passes through a 2.0 mm divergence slit followed by a 0.2 mm anti-scatter slit and knife edge. The diffracted beam passes through an 8.0 mm receiving slit with 2.5° Soller slits followed by the Lynxeye Detector. The software used for data collection and analysis was Diffrac Plus XRD Commander and Diffrac Plus EVA respectively.

Samples were run under ambient conditions as flat plate specimens using powder as received. The sample was prepared on a polished, zero-background (510) silicon wafer by gently pressing onto the flat surface or packed into a cut cavity. The sample was rotated in its own plane.

The details of the standard data collection method are:
Angular range: 2 to 42° 2θ
Step size: 0.05° 2θ
Collection time: 0.5 s/step (total collection time: 6.40 min)

The instrument is performance checked weekly using NIST1976 corundum to the peak position of 35.149±0.01° 2θ

### 1.2 Differential Scanning Calorimetry (DSC)

DSC data were collected on a TA Instruments Q2000 equipped with a 50 position auto-sampler. Typically, 0.5-3 mg of each sample, in a pin-holed aluminium pan, was heated at 10° C./min from 25° C. to 300° C. A purge of dry nitrogen at 50 ml/min was maintained over the sample.

The instrument control software was Advantage for Q Series and Thermal Advantage and the data were analysed using Universal Analysis or TRIOS.

### 1.3 Thermo-Gravimetric Analysis (TGA)

TGA data were collected on a TA Instruments Discovery TGA, equipped with a 25 position auto-sampler. Typically, 5-10 mg of each sample was loaded onto a pre-tared aluminium DSC pan and heated at 10° C./min from ambient temperature to 400° C. A nitrogen purge at 25 ml/min was maintained over the sample.

The instrument control software was TRIOS and the data were analysed using TRIOS or Universal Analysis.

### 1.4 Gravimetric Vapour Sorption (GVS)

Hygroscopicity of a solid material may be determined by means of gravimetric vapour sorption (GVS) analysis, sometimes known by dynamic vapour sorption (DVS) analysis.

The experiment subjects a sample material which is held in a fine wire basket on a microbalance within a temperature and humidity controlled environment (chamber). Using the software, the collected data can then be processed to determine the isotherm points at the increment ranges specified during the experiment and show the overall water uptake of the material.

Sorption isotherms were obtained using a SMS DVS Intrinsic moisture sorption analyser, controlled by DVS Intrinsic Control software. The sample temperature was maintained at 25° C. by the instrument controls. The humidity was controlled by mixing streams of dry and wet nitrogen, with a total flow rate of 200 ml/min. The relative humidity was measured by a calibrated Rotronic probe (dynamic range of 1.0-100% RH), located near the sample. The weight change, (mass relaxation) of the sample as a function of %RH was constantly monitored by a microbalance (accuracy ±0.005 mg).

Typically, 5-30 mg of sample was placed in a tared mesh stainless steel basket under ambient conditions. The sample was loaded and unloaded at 40% RH and 25° C. (typical room conditions). A moisture sorption isotherm was performed as outlined below (2 scans per complete cycle). The standard isotherm was performed at 25° C. at 10% RH intervals over a 0-90% RH range. Typically, a double cycle (4 scans) was carried out. Data analysis was carried out within Microsoft Excel using the DVS Analysis Suite.

TABLE 1
Method for SMS DVS Intrinsic experiments

| Parameter | Value |
|---|---|
| Adsorption - Scan 1 | 40-90 |
| Desorption, Adsorption - Scan 2 | 90-0, 0-40 |
| Intervals (% RH) | 10 |
| Number of Scans | 4 |
| Flow rate (ml/min) | 200 |
| Temperature (° C.) | 25 |
| Stability (° C./min) | 0.2 |
| Sorption Time (hours) | 6 hour time out |
| Number of cycles | 2 |

### Abbreviations

TBME tert-butyl methyl ether
2-Methyl THF 2-methyl tetrahydrofuran
DCM dichloromethane
DMF dimethylformamide
DMSO dimethyl sulfoxide
IPA isopropanol
MEK methyl ethyl ketone
MIBK methyl isobutyl ketone
NMP 1-methyl-2-pyrrolidinone
RT room temperature
% RH percent Relative Humidity
THF tetrahydrofuran

### Amorphous Avapritinib

### Example 1

Avapritinib (ca 30 mg) was dissolved in 1,4-dioxane (900 μl, 30 vol) in a vial and the solution was flash cooled in a dry ice/acetone bath and lyophilised on a freeze drier. Once dry, the solid was analysed by XRPD.

### Anhydrous Avapritinib

### Examples 2-10

Avapritinib anhydrate was prepared according to following general example: Avapritinib (x mg) was dissolved in a solvent (y μl, z vol) at 50° C. The solution was stirred at 50° C. for 1 hour before cooling to 5° C. at 0.1° C./min and stirred overnight. The resulting suspension was filtered and dried under suction before analysis by XRPD.

| | Mass of | | | |
|---|---|---|---|---|
| | avapritinib | | Volume of solvent | |
| Example | (x mg) | Solvent | y μl | z vol. |
| 2 | 28.8 | Acetone | 1800 | 60 |
| 3 | 29.6 | DMSO | 150 | 5 |
| 4 | 29.3 | Ethyl acetate | 2400 | 80 |
| 5 | 29.5 | MEK | 1200 | 40 |
| 6 | 29.4 | 2-methyl THF | 1800 | 60 |
| 7 | 29.0 | DCM | 300 | 10 |
| 8 | 28.6 | Nitromethane | 1200 | 40 |
| 9 | 29.1 | 1,2-Dimethoxyethane | 1200 | 40 |
| 10 | 29.2 | Acetone/water | 1200 | 40 |
| | | (95:5 v/v) | | |

### Example 11

Avapritinib (100.9 mg) was dissolved in acetone/water (90:10 v/v) (2 ml, 20 vol) at 50° C. The solution was stirred at 50° C. for 1 hour before cooling to 5° C. at 0.1° C./min and further stirred for 4 days. The resulting suspension was filtered and dried under suction to provide anhydrous avapritinib before analysis by XRPD.

### Example 12

Avapritinib (501.2 mg) was dissolved in acetone/water (90:10 v/v) (10 ml, 20 vol) at 50° C. The solution was stirred at 50° C. for 1 hour before cooling to 5° C. at 0.1° C./min and further stirred for 4 days at 5° C. The resulting suspension was filtered and dried under suction to provide anhydrous avapritinib before analysis by XRPD. Yield: 63.1%

### Examples 13-23

Avapritinib anhydrate was prepared according to following general example: Avapritinib (x mg) was suspended with a solvent (3000 μl, 100 vol) at 50° C. The suspension was shaken at 50° C. overnight. The resulting suspension was filtered and dried under suction before analysis by XRPD.

| | Mass of | |
|---|---|---|
| | avapritinib | |
| Example | (x mg) | Solvent |
| 13 | 28.7 | TBME |
| 14 | 29.5 | Ethanol |
| 15 | 29.2 | Heptane |
| 16 | 29.2 | Isopropyl acetate |
| 17 | 29.0 | MIBK |
| 18 | 29.0 | IPA |
| 19 | 29.0 | Acetonitrile |
| 20 | 29.0 | Toluene |
| 21 | 29.4 | 2-Methyl-1-propanol |
| 22 | 29.0 | 1-Propanol |
| 23 | 29.2 | Ethanol/water (95:5 v/v) |

### Examples 24-26

Avapritinib anhydrate was prepared according to following general example: Avapritinib (x mg) was dissolved in a solvent (y μl, z vol) at 50° C. The solution was stirred at 50° C. for 1 hour before cooling to 5° C. at 0.1° C./min and further stirred overnight. The resulting solution was left uncapped to evaporate. The resulting solid was analysed by XRPD.

| | Mass of | | | |
|---|---|---|---|---|
| | avapritinib | | Volume of solvent | |
| Example | (x mg) | Solvent | y μl | z vol. |
| 24 | 29.1 | DMF | 150 | 5 |
| 25 | 29.1 | NMP | 150 | 5 |
| 26 | 29.5 | 2-Methoxyethanol | 600 | 20 |

### Examples 27-35

Avapritinib anhydrate was prepared according to following general example: Avapritinib (x mg) was dissolved in a solvent (y μl, z vol) at 25° C. and the solution was left uncapped to evaporate. Once dry, the solid was analysed by XRPD.

| | Mass of | | | |
|---|---|---|---|---|
| | avapritinib | | Volume of solvent | |
| Example | (x mg) | Solvent | y μl | z vol. |
| 27 | 29.1 | Acetone | 3000 | 100 |
| 28 | 28.9 | MEK | 2400 | 80 |
| 29 | 29.1 | 2-Methyl THF | 3000 | 100 |
| 30 | 29.1 | DCM | 300 | 10 |
| 31 | 29.3 | DMF | 150 | 5 |
| 32 | 28.9 | Nitromethane | 2400 | 80 |
| 33 | 29.2 | 2-Methoxyethanol | 600 | 20 |
| 34 | 29.2 | 1,2-Dimethoxyethane | 1800 | 60 |
| 35 | 29.1 | Acetone/water | 1200 | 40 |
| | | (95:5 v/v) | | |

### Example 36

Avapritinib anhydrate was prepared according to following example: Avapritinib (28.9 mg) was suspended with ethyl acetate (3 ml, 100 vol) at 25° C. and the suspension was left to stir at 25° C. overnight. The resulting suspension was filtered and dried under suction before analysis by XRPD.

### Examples 37-59

Avapritinib anhydrate was prepared according to following general example: Avapritinib (x mg) was dissolved in a first solvent (y μl, z vol) and left to stir for 5 minutes at 50° C. After 5 minutes of stirring, the sample was suspended with a second solvent (z μl). The resulting solution was then stirred at 50° C. for 1 hour before cooling to 5° C. at 0.1° C./min and further stirred overnight. The resulting suspension was filtered and dried under suction before analysis by XRPD.

| | Mass of | | Volume of | | | |
|---|---|---|---|---|---|---|
| | avapritinib | First | solvent | | Second | |
| Example | (mg) | solvent | y μl | z vol. | solvent | z μl |
| 37 | 29.1 | DMSO | 150 | 5 | TBME | 750 |
| 38 | 29.3 | DMSO | 150 | 5 | Ethanol | 750 |
| 39 | 29.2 | DMSO | 150 | 5 | Isopropyl | 750 |
| | | | | | acetate | |
| 40 | 29.0 | DMSO | 150 | 5 | Water | 150 |
| 41 | 29.3 | DMSO | 150 | 5 | Acetonitrile | 750 |
| 42 | 29.3 | DMF | 150 | 5 | TBME | 750 |
| 43 | 29.3 | DMF | 150 | 5 | Ethanol | 750 |
| 44 | 29.0 | DMF | 150 | 5 | Isopropyl | 750 |
| | | | | | acetate | |
| 45 | 29.3 | DMF | 150 | 5 | Water | 150 |
| 46 | 29.1 | DMF | 150 | 5 | Acetonitrile | 750 |
| 47 | 29.0 | DMF | 150 | 5 | Toluene | 750 |
| 48 | 29.0 | DCM | 300 | 10 | TBME | 300 |
| 49 | 29.0 | DCM | 300 | 10 | Ethanol | 1500 |
| 50 | 28.9 | DCM | 300 | 10 | n-Heptane | 300 |
| 51 | 29.0 | DCM | 300 | 10 | Isopropyl | 1500 |
| | | | | | acetate | |
| 52 | 29.0 | DCM | 300 | 10 | Acetonitrile | 1500 |
| 53 | 29.0 | DCM | 300 | 10 | Toluene | 1500 |
| 54 | 29.3 | 1,4- | 300 | 10 | TBME | 1500 |
| | | Dioxane | | | | |
| 55 | 29.1 | 1,4- | 300 | 10 | Ethanol | 1500 |
| | | Dioxane | | | | |
| 56 | 29.1 | 1,4- | 300 | 10 | Isopropyl | 1500 |
| | | Dioxane | | | acetate | |
| 57 | 29.0 | 1,4- | 300 | 10 | Water | 600 |
| | | Dioxane | | | | |
| 58 | 29.0 | 1,4- | 300 | 10 | Acetonitrile | 1500 |
| | | Dioxane | | | | |
| 59 | 29.0 | 1,4- | 300 | 10 | Toluene | 1500 |
| | | Dioxane | | | | |

### Example 60-76

Avapritinib anhydrate was prepared according to following general example: Amorphous avapritinib (˜30 mg) was suspended with a solvent (300 μl, 10 vol) and left to stir at 5° C. for 7 days. An aliquot of the resulting suspension was analysed by XRPD.

| Example | Solvent |
|---|---|
| 60 | Acetone |
| 61 | Ethanol |
| 62 | Ethyl acetate |
| 63 | Heptane |
| 64 | Isopropyl acetate |
| 65 | MEK |
| 66 | MIBK |
| 67 | IPA |
| 68 | Water |
| 69 | Acetonitrile |
| 70 | 2-Methyl THF |
| 71 | Nitromethane |
| 72 | 2-Methyl-1-propanol |
| 73 | 1-Propanol |
| 74 | 1,2-Dimethoxyethane |
| 75 | Acetone/water (95:5 v/v) |
| 76 | Ethanol/water (95:5 v/v) |

### Example 77-95

Avapritinib anhydrate was prepared according to following general example: Amorphous avapritinib (˜30 mg) was suspended with a solvent (300 μl, 10 vol) and left to shake at 50° C. for 7 days. An aliquot of the resulting suspension was analysed by XRPD.

| Example | Solvent |
|---|---|
| 77 | Acetone |
| 78 | TBME |
| 79 | Ethanol |
| 80 | Ethyl acetate |
| 81 | Heptane |
| 82 | Isopropyl acetate |
| 83 | MEK |
| 84 | MIBK |
| 85 | IPA |
| 86 | Water |
| 87 | Acetonitrile |
| 88 | 2-Methyl THF |
| 89 | Toluene |
| 90 | Nitromethane |
| 91 | 2-Methyl-1-propanol |
| 92 | 1-Propanol |
| 93 | 1,2-Dimethoxyethane |
| 94 | Acetone/water (95:5 v/v) |
| 95 | Ethanol/water (95:5 v/v) |

### Example 96

Avapritinib anhydrate was prepared according to following general example:

A stock solution of avapritinib (240.3 mg) was dissolved in THF (4.8 ml, 20 vol) with sonication.

For each of Examples 96-102: 600 μl of avapritinib solution was pipetted into a smaller vial and left to stir for 5 minutes at 60° C. After 5 minutes of stirring, the sample was suspended with a second solvent (x μl). The resulting solution was then stirred at 50° C. for 1 hour before cooling to 5° C. at 0.1° C./min and further stirred overnight. The resulting suspension was filtered and dried under suction before analysis by XRPD.

| Example | Second solvent | x μl |
|---|---|---|
| 96 | TBME | 3000 |
| 97 | Ethanol | 3000 |
| 98 | Heptane | 1200 |
| 99 | Isopropyl acetate | 3000 |
| 100 | Water | 1200 |
| 101 | Acetonitrile | 3000 |
| 102 | Toluene | 3000 |

### Example 103

Avapritinib (2 g) was dissolved in THF (20 ml, 10 vol) at 60° C. The solution was cooled to 50° C. at 0.25° C./min and the resulting cloudy solution was seeded with anhydrous avapritinib prepared according to Example 12. The sample was cooled further to 25° C. at 0.25° C./min then heptane (20 ml) was added. The thick suspension was cooled to 5° C. at 0.25° C./min and stirred at 5° C. overnight. The resulting suspension was filtered and dried under vacuum at RT overnight. Yield: 93.7%.

### Avapritinib Methanol Solvate

### Example 104

Avapritinib (29.0 mg) was suspended with methanol (3000 μl, 100 vol) at 50° C. The suspension was shaken at 50° C. overnight. The resulting suspension was filtered and dried under suction before analysis by XRPD.

### Example 105

Avapritinib (99.6 mg) was suspended with methanol (4 ml, 40 vol) at 50° C. The suspension was matured between RT and 50° C. (4 hours at each temperature under shaking) for 4 days. The resulting suspension was filtered and dried under suction before analysis by XRPD.

### Example 106

Amorphous avapritinib (˜30 mg) was suspended with methanol (300 μl, 10 vol) and stirred to stir at 5° C. for 7 days. An aliquot of the resulting suspension was analysed by XRPD.

### Example 107

Amorphous avapritinib (˜30 mg) was suspended with methanol (300 μl, 10 vol) and stirred to shaken at 50° C. for 7 days. An aliquot of the resulting suspension was analysed by XRPD.

### Example 108

Avapritinib (500.1 mg) was suspended with methanol (10 ml, 20 vol) at 50° C. The suspension was matured between RT and 50° C. (4 hours at each temperature under shaking) for 4 days. The resulting suspension was filtered and dried under suction before analysis by XRPD.

### Example 109

Avapritinib (2 g) charged into a 25 ml vessel with overhead stirring and suspended with methanol (10 ml, 5 vol). The resulting paste was too thick for sufficient stirring at 750 rpm and suspended with further methanol (+10 ml, 5 vol). The thick suspension was stirred at 25° C. for 1 hour then cooled to 5° C. at 0.25° C./min and stirred at 5° C. overnight. The resulting sample became thicker leaving only a wet solid. The wet solid was transferred from the vessel to a filter funnel and additional methanol used to wash the solids.

### Avapritinib Hydrate

### Example 110

Avapritinib methanol solvate prepared according to Example 105 was stored at 40° C./75% RH for 7 days.

### Example 111

Avapritinib methanol solvate prepared according to Example 108 was left open to ambient conditions overnight.

### Example 112

Avapritinib methanol solvate prepared according to Example 108 was stored at 40° C./75% RH for 7 days.

### Example 113

Avapritinib methanol solvate prepared according to Example 108 was stored at 25° C./97% RH for 7 days.

### Example 114

Avapritinib methanol solvate prepared according to Example 108 was slurried in water (500 μl, 10 vol) at RT overnight. The resulting suspension was filtered and dried under suction before analysis by XRPD.

### Example 115

Avapritinib methanol solvate prepared according to Example 109 was dried under vacuum at RT for 4 days. Yield: 86.1% (molar yield).

1. A polymorph of avapritinib, which is amorphous avapritinib.

2. A process for preparing amorphous avapritinib, the process comprising the steps of:
(a) dissolving avapritinib in a suitable solvent to form a solution of avapritinib;
(b) flash cooling the solution of avapritinib; and
(c) quickly removing the solvent to form the amorphous avapritinib.

3. A crystalline form of avapritinib which is crystalline avapritinib anhydrate and which is free or substantially free of other polymorphic forms of avapritinib.

4. A crystalline form according to claim 3, wherein the crystalline avapritinib anhydrate has an X-ray powder diffraction pattern comprising one or more peaks selected from the group consisting of about 3.8, 7.6, 10.0, 11.5, 13.8, 15.3, 16.7, 18.0, 19.1, 19.9, 20.2, 21.4, 22.9, 23.7, 25.1, 25.7, 26.0, 27.7, and 30.6 degrees two-theta±0.2 degrees two-theta.

5. A crystalline form according to claim 2, wherein the X-ray powder diffraction pattern substantially as shown in FIG. 2.

6. A crystalline form according to any one of claims 3 to 5, which has a DSC thermogram comprising an endothermic event with an onset at about 192.6° C.

7. A crystalline form according to claim 6, which has a DSC thermogram substantially as shown in FIG. 3.

8. A crystalline form according to any one of claims 3 to 7, which has a TGA thermogram comprising no substantially mass loss when heated from about ambient temperature to about 200° C.

9. A crystalline form according to claim 8, which has a TGA thermogram substantially as shown in FIG. 3.

10. A process for preparing crystalline avapritinib anhydrate, the process comprising the steps of:
(a) contacting avapritinib with a solvent selected from the group consisting of acetone, dimethyl sulfoxide, ethyl acetate, methyl ethyl ketone, 2-methyl tetrahydrofuran, dichloromethane, nitromethane, 1,2-dimethyoxyethane, water, tert-butyl methyl ether, ethanol, heptane, isopropyl acetate, methyl isobutyl ketone, isopropanol, acetonitrile, toluene, 2-methyl-1-propanol, 1-propanol, ethanol, dimethylformamide, 1-methyl-2-pyrrolidinone, 2-methoxyethanol, and combinations thereof;
(b) forming a solution or suspension of avapritinib in the solvent; and
(c) recovering avapritinib anhydrate as a crystalline solid.

11. A crystalline form of avapritinib which is crystalline avapritinib methanol solvate.

12. A crystalline form of avapritinib according to claim 11, wherein the crystalline avapritinib methanol solvate has an X-ray powder diffraction pattern comprising one or more peaks selected from the group consisting of about 5.2, 9.3, 10.4, 11.9, 13.7, 14.6, 16.1, 17.5, 18.7, 20.8, 21.4, 23.9, 24.6, 25.4, and 25.7 degrees two-theta±0.2 degrees two-theta.

13. A crystalline form of avapritinib according to claim 12, which has the X-ray powder diffraction pattern substantially as shown in FIG. 5.

14. A crystalline form of avapritinib according to any one of claims 11 to 13, wherein the crystalline avapritinib methanol solvate has a DSC thermogram comprising two endothermic events with onset temperatures of about 72.5° C. and about 191.4° C.

15. A crystalline form of avapritinib according to claim 14, which has a DSC thermogram substantially as shown in FIG. 6.

16. A crystalline form of avapritinib according to any one of claims 11 to 15, wherein the crystalline avapritinib methanol solvate a TGA thermogram comprising about 5.5% mass loss when heated from about ambient temperature to about 200° C.

17. A crystalline form of avapritinib according to claim 16, which has a TGA thermogram substantially as shown in FIG. 6.

18. A process for preparing avapritinib methanol solvate, the process comprising the steps of:
(a) contacting avapritinib with methanol; and
(b) forming a suspension of avapritinib in methanol.

19. A process according to claim 18, which further comprises the step of recovering avapritinib methanol solvate as a crystalline solid.

20. A crystalline form of avapritinib which is crystalline avapritinib hydrate and which is free or substantially free of other polymorphic forms of avapritinib.

21. A crystalline form of avapritinib according to claim 20, wherein the crystalline avapritinib hydrate has an X-ray powder diffraction pattern comprising one or more peaks selected from the group consisting of about 5.3, 9.3, 10.4, 10.7, 12.0, 13.9, 14.7, 15.2, 16.1, 17.4, 17.9, 18.7, 18.9, 20.8, 21.4, 22.4, 22.7, 23.3, 23.9, 24.6, 25.6, 27.2, 28.4, and 29.7degrees two-theta±0.2 degrees two-theta.

22. A crystalline form of avapritinib according to claim 21, wherein the crystalline avapritinib hydrate has an X-ray powder diffraction pattern substantially as shown in FIG. 7.

23. A crystalline form of avapritinib according to any one of claims 20 to 22, wherein hydrate crystalline avapritinib hydrate has a DSC thermogram comprising two endothermic events with onset temperatures of about 29.8° C. and about 191.3° C.

24. A crystalline form of avapritinib according to claim 23, wherein the crystalline avapritinib hydrate has a DSC thermogram substantially as shown in FIG. 8.

25. A crystalline form of avapritinib according to any one of claims 20 to 24, wherein hydrate crystalline avapritinib hydrate has a TGA thermogram comprising about 3.5% mass loss when heated from about ambient temperature to about 200° C.

26. A crystalline form of avapritinib according to claim 25, wherein the crystalline avapritinib hydrate has a TGA thermogram substantially as shown in FIG. 8.

27. A process for preparing crystalline avapritinib hydrate, the process comprising the step of hydrating crystalline avapritinib methanol solvate.

28. A pharmaceutical composition comprising avapritinib and a pharmaceutically acceptable excipient,
wherein the avapritinib is selected from the group consisting of (i) amorphous avapritinib, (ii) crystalline avapritinib anhydrate which is free or substantially free of other polymorphic forms of avapritinib, and (iii) crystalline avapritinib hydrate which is free or substantially free of other polymorphic forms of avapritinib.

29. A method for treating cancer in a patient comprising administering a therapeutically effective amount of avapritinib to the patient,
wherein the avapritinib is selected from the group consisting of (i) amorphous avapritinib, (ii) crystalline avapritinib anhydrate which is free or substantially free of other polymorphic forms of avapritinib, and (iii) crystalline avapritinib hydrate which is free or substantially free of other polymorphic forms of avapritinib.

30. A method for treating cancer according to claim 29, wherein the cancer is gastrointestinal stromal tumours.

31. Avapritinib for use in treating cancer, wherein the avapritinib is selected from the group consisting of (i) amorphous avapritinib, (ii) crystalline avapritinib anhydrate which is free or substantially free of other polymorphic forms of avapritinib, and (iii) crystalline avapritinib hydrate which is free or substantially free of other polymorphic forms of avapritinib.

32. Avapritinib for use in treating cancer according to claim 31, wherein the cancer is gastrointestinal stromal tumours.

33. A method for treating mastocytosis in a patient comprising administering a therapeutically effective amount of avapritinib to the patient,
wherein the avapritinib is selected from the group consisting of (i) amorphous avapritinib, (ii) crystalline avapritinib anhydrate which is free or substantially free of other polymorphic forms of avapritinib, and (iii) crystalline avapritinib hydrate which is free or substantially free of other polymorphic forms of avapritinib.

34. Avapritinib for use in treating mastocytosis, wherein the avapritinib is selected from the group consisting of (i) amorphous avapritinib, (ii) crystalline avapritinib anhydrate which is free or substantially free of other polymorphic forms of avapritinib, and (iii) crystalline avapritinib hydrate which is free or substantially free of other polymorphic forms of avapritinib.
